(19) United States
(12) Patent Application Publication
(10) Pub. No.: US 2025/0363340 A1
(43) Pub. Date: Nov. 27, 2025

(54) NODE SCALE-ADAPTIVE NEURON SPIKE SORTING METHOD BASED ON NEUROMORPHIC COMPUTING

(71) Applicant: ZHEJIANG UNIVERSITY, Hangzhou (CN)

(72) Inventors: YU QI, HANGZHOU (CN); GANG PAN, HANGZHOU (CN); HANG YU, HANGZHOU (CN)

(21) Appl. No.: 19/214,280

(22) Filed: May 21, 2025

(30) Foreign Application Priority Data
May 27, 2024 (CN) 2024106616580

(51) Int. Cl. G06N 3/049; G06N 3/065

(52) U.S. Cl. CPC G06N 3/049; G06N 3/065

(57) ABSTRACT

The present invention discloses a node scale-adaptive neuron spike sorting method based on neuromorphic computing, and relates to the field of electroencephalogram signal spike sorting and decoding, the present invention proposes a spiking neural network framework comprising a two-layer spiking neural network and an attention neuron node, by incorporating prior knowledge of spike waveforms, this method automatically guides the addition and removal of network nodes to optimize computational resource allocation according to specific requirements, thereby minimizing hardware resource wastage. This method is characterized by low hardware overhead, high computational speed, and high consistency of results across different datasets. This method enhances the speed of spike sorting processes and shows potential for providing fully automated neuronal classification technology support for wireless implantable brain signal acquisition devices.

### FIELD OF TECHNOLOGY

The present invention relates to the field of brain signal spike sorting and decoding, in particular to a node scale-adaptive neuron spike sorting method based on neuromorphic computing.

### BACKGROUND TECHNOLOGY

Spike sorting plays an important role in neuroscience research by accurately classifying spike events generated by individual neurons recorded in signal channels. By accurately identifying and sorting these neurons, spike sorting technology provides valuable insights into the functional characteristics, connectivity patterns, and temporal dynamics of specific neuron types. These information is crucial for understanding neural circuits and unraveling the complexity of brain function.

The traditional spike sorting method mainly relies on manual inspection or semi-automatic processes. However, the latest developments in microelectronics and nanoscale structures have enabled neural recording to use thousands of channels for large-scale recording, it is almost impossible to manually spike sorting. Large scale recording can also lead to critical issues in computation and signal transmission. The increase in data scale has led to more signal processing and transmission costs, making it almost impossible for brain implant devices to achieve.

For applications that primarily require spike training signals, chip based spike sorting is considered a potential solution to reduce transmission bandwidth. As the name suggests, these sorting methods are located inside brain implant devices and only transmit spike events to downstream tasks. Through this method, the size of transmitted data can be greatly reduced, and wireless transmission is expected to be achieved. Therefore, the key issue lies in a low-cost automatic spike sorting method that can be applied to large-scale recording for brain implantation calculations.

Although seemingly uncomplicated, fully automating spike sorting at low cost remains a challenging task. Previously, some methods have been adopted to transmit the entire or partial sorting results. These methods range from analyzing independent action potentials to attempting spike extraction and classification. For example, the Chinese patent document with the publication number CN115844422A discloses a method for neuronal spike sorting, which includes obtaining raw spike signals and preprocessing the obtained spike signals; using the heuristic adaptive threshold to perform spike detection on the preprocessed dataset, a spike signal dataset is obtained; reducing the dimensionality of the spike data using principal component analysis to obtain eigenvalues and eigenvectors, and mapping the spike points to the feature space constructed by the eigenvectors; using feature values and spike data as inputs for K-means clustering, continuously iterating to ensure that the clustering center does not change while ensuring that each sample is closest to its corresponding class center, the classification result is obtained.

The recent progress in large-scale spike sorting technology has demonstrated high accuracy while operating in a completely autonomous manner. However, these methods often face the problem of high computational complexity, making it difficult to achieve fast channel expansion within the power consumption limitations of implanted devices.

Currently, these methods face two major obstacles. Firstly, the implementation of complex task algorithms incurs significant hardware costs, requiring higher demands for computing resources to be met within the limited space of the device. Secondly, the temperature sensitivity and rejection response of nerve cells require implanted devices to have low power consumption and small size. Reaching a suitable balance between power consumption and circuit area is a key area that requires further exploration in this field.

### SUMMARY OF THE INVENTION

The present invention provides a node scale-adaptive neuron spike sorting method based on neuromorphic computing, which improves the problems of slow manual classification speed, inconsistent classification results from different experts, and long time required in spike potential signal classification. This method improves the speed of spike sorting process to a certain extent and maintains high classification consistency on different datasets. In addition, this method is also helpful for the deployment of implanted chips.

A node scale-adaptive neuron spike sorting method based on neuromorphic computing, comprising the following steps:
(1) obtaining original multi-channel neural signals, removing low-frequency local field potentials through a band-pass filter, and performing whitening preprocessing and artifact removal on each channel's neural signals;
(2) detecting and aligning candidate spike on each signal channel, specifically by using a nonlinear energy operator to calculate the energy intensity of each position in the discrete signal, wherein a time window exceeding the threshold is determined as a candidate spike, and then aligning the candidate spike based on the spike position;
(3) constructing a spiking neural network framework, wherein the framework comprises a two-layer spiking neural network and an attention neuron node; among them, the first layer of the spiking neural network is a perception layer and the second layer of the spiking neural network is a cognitive layer, each neuron on the two-layer connects in a fully connected manner and dynamically updates the connecting synapses; the attention neuron is unidirectionally connected to control all neurons in the network;
(4) inputting the aligning candidate spike into the spiking neural network framework, wherein the perception layer of the spiking neural network is used to pulse code the candidate spike, and the discrete signal of each time point of the candidate spike potentials is mapped to a group of pulse sequences in the form of Gaussian Receptive field coding;

The neurons on the cognitive layer respond to different spike sequence inputs, and update the connecting synapses between the activated neurons with the corresponding neurons in the perception layer based on the winner-take-all mechanism; when the cumulative voltage of neurons in the cognitive layer exceeds the voltage threshold, the pulse sequences are output as a time stamp sequence in response to the action potential of different cells;

The attention neuron node responds to the waveform prior knowledge of input candidate spike, modifies the waveform of input candidate spike potential, and controls the threshold changes of addition strategy, deletion and merging strategy of perception layer nodes;
(5) for the original neural signals corresponding to the time stamp sequence, dividing the spike and noise according to a threshold, and cognitive layer nodes dynamically updating the threshold by pulse waveform prior knowledge, and each channel reconstructs waveforms from different cells based on the time stamp sequence output by the spiking neural network.

Furthermore, in the step (1), the band-pass filter adopts a 3rd order Butterworth filter with a band-pass frequency of 300-3000 Hz.

In the step (2), using a nonlinear energy operator to calculate the energy intensity of each position in the discrete signal, the formula is:

$$ψ [ x ( n ) ] = {x}^{2} ( n ) - x ( n + 1 ) \cdot x ( n - 1 )$$

wherein, x(n) is the sampling point of the n time waveform, the threshold is set to 0.05, and the first 25 and last 38 energy operators that exceed the threshold are considered as candidate spike at a total of 64 time points when the window is cut off.

In the step (2), when aligning the candidate spike based on the spike position, the spike maximum peak position is first interpolated through upsampling, and after realignment, the waveform is downsampled to its original length.

In the step (4), the form of Gaussian Receptive field coding is as follows:

$${J}_{( t , m , n )} = {𝕡}_{μ , θ} ( {S}_{t} )$$

wherein, μ is the central position of neurons in the Receptive field, θ is the width of neurons in the Receptive field, Sₜ is the signal sequence at time t, J_{(t,m,n)} is the pulse firing of the neurons (m, n) in the perception layer at time t, and [symbol] is the Poisson process of the Gaussian Receptive field.

In the step (4), the winner-take-all mechanism is as follows: when a neuron is activated, other neurons are suppressed and will not be updated, only the weight of connecting synapses between the activated neuron with the neurons in the perception layer is enhanced or reduced;
wherein, when updating the connecting synapses between the activated neurons with the corresponding neurons in the perception layer, the neuron selection method is as follows:

$$\dot{ϵ} = {max}_{ϵ} z ( ϵ , t )$$

wherein, \dot{ϵ} is the neurons in the cognitive layer for selected execution updates, z(ϵ, t) is the voltage value of neurons in the cognitive layer at time t;
a weight update method of the connecting synapses between the two layers is as follows:

$${\hat{ω}}_{t + 1} = STDP ( {ω}_{t} , {τ}_{+} , {τ}_{-} )$$

wherein, \hat{ω}ₜ₊₁ is the synaptic weight at t+1 time after update, ωₜ is the synaptic weight at time t before the update, τ₊ is the constant for postsynapses firing, τ₋ is the constant for presynapses firing.

In the step (4), the attention neuron node responds to the waveform prior knowledge of input candidate spike, modifies the waveform of input candidate spike potential, a generating method for a waveform masking [symbol] ₜ is as follows:

$${ℳ}_{t} = {S}_{t} \cdot {G}_{t}$$

among them, Sₜ represents the signal sequence at time t, Gₜ denotes the waveform modification mask; the generation method of the waveform modification mask Gₜ is as follows:

$${G}_{t} = \{ \begin{matrix}\frac{{l}_{AP} - {t}_{A 1} + t}{{l}_{AP}} , & {t}_{A 1} - {l}_{AP} \leq t < {t}_{A 1} , \\ 1 , & {t}_{A 1} \leq t \leq {t}_{A 2} , \\ 1 - \frac{t - {l}_{A 2}}{{l}_{TP}} , & {t}_{A 2} < t \leq {t}_{A 2} + {l}_{TP} , \\ 0 , & other locations .\end{matrix}$$

among them, l_{AP} represents the action spike potential width, t_{A1} and t_{A2} are the time points of the pre-hyperpolarization peak and post-hyperpolarization peak, respectively, and l_{TP} denotes the trough-to-peak duration of the spike.

In the step (4), when implementing the addition strategy of perception layer nodes: if the difference between the masked waveform of the input spike and the stored waveform in the network is smaller than the similarity threshold Thₛᵢₘ, a new node is added to the perception layer, the perception layer node update comparison method is:

$$\frac{‖ {ℳ}_{t} - ( Φ ∩ {ω}_{ϵ} ) ‖}{‖ Φ∩ {ω}_{ϵ} ‖} < T {h}_{s i m}$$

among them, [symbol] ₜ is the masked waveform, ω_{ϵ} represents the connection weights between the selected node ϵ in the perception layer and the previous layer, and Φ is an all-ones matrix with the same dimensions as the weight matrix.

In the step (4), when implementing the deletion and merging strategy of perception layer nodes: if the difference between stored waveforms in the network is smaller than the similarity threshold Thₛᵢₘ, the two nodes are merged, the perception layer node update comparison method is:

$$\frac{‖ {ω}_{i} - {ω}_{j} ‖}{‖ \sqrt{{ω}_{i}} ‖ ‖ \sqrt{{ω}_{j}} ‖} < T {h}_{s i m}$$

among them, ωᵢ and ωⱼ are the connection weights corresponding to distinct nodes in the perception layer.

In the step (4), when updating thresholds during perception layer node strategy adjustments, the similarity threshold Thₛᵢₘ is updated as:

$$T {h}_{s i m} = α \cdot ( 1 + floor ( \frac{K}{β} ) )$$

among them, α is the scaling control coefficient, β is the waveform count control coefficient, and K is the input spike waveform iteration count;
an output threshold [symbol] ₒᵤₜₚᵤₜ is updated as:

$${o u t p u t}_{} = T {h}_{o u t p u t} + sign ( {z}_{( t , ζ )} - T {h}_{o u t p u t} ) \cdot ( 1 + floor ( \frac{K}{β} ) )$$

where [symbol] ₒᵤₜₚᵤₜ is the updated threshold for the next deletion strategy iteration, z_{(t,ζ)} is the voltage value of node ζ in the perception layer at time t, β is the waveform count control coefficient, and K is the input spike waveform iteration count.

Compared with the prior art, the present invention has the following beneficial effects:
1. The present invention has the effect of adaptive node size through network addition and deletion strategy, which can achieve the minimum hardware overhead under the same computing.
2. The present invention has strong recognition universality by integrating neural prior knowledge, and can adapt to brain signal acquisition scenarios with more signal channels.
3. The present invention is expected to achieve online closed-loop brain computer interface spike potential classification by deploying on a neuromorphic chip with ultra-low power hardware overhead.

### BRIEF DESCRIPTION OF THE DRAWINGS

FIG. 1 is a paradigm flowchart of a node scale-adaptive neuron spike sorting method based on neuromorphic computing of the present invention;

FIG. 2 shows the effect of the method of the present invention on a simulated dataset.

### DETAILED DESCRIPTION OF THE EMBODIMENTS

The following is a further detailed description of the present invention in conjunction with the accompanying drawings and embodiments. It should be noted that the embodiments described below are intended to facilitate the understanding of the present invention without any limiting effect.

This embodiment uses a simulated dataset where two spike potential waveforms with distinct shapes are selected from a real spike library, these spikes potentials are fired at different frequency probabilities, and mechanical noise along with distant spike potential noise is incorporated to simulate real experimental datasets.

Although the network can autonomously learn the emergence and transitions of spikes potentials, it requires predefined hyper-parameters to configure the network structure strategy before operation. A well-tuned set of parameters enables the network to dynamically add or delete nodes based on varying waveform inputs, avoiding both over-clustering and misclassification. The adopted parameters are as follows: α: scaling control coefficient 0.04; β: waveform count control coefficient 10; Iₘₐₓ: upper limit of Receptive field 200; Iₘᵢₙ: lower limit of Receptive field—200; γ: field neuron form factor 2; dᵣ: the average distance between adjacent Receptive field 13; τ₊: short term plasticity time presynaptic constant 0.2; τ₋: short term plasticity time postsynaptic constant 0.1.

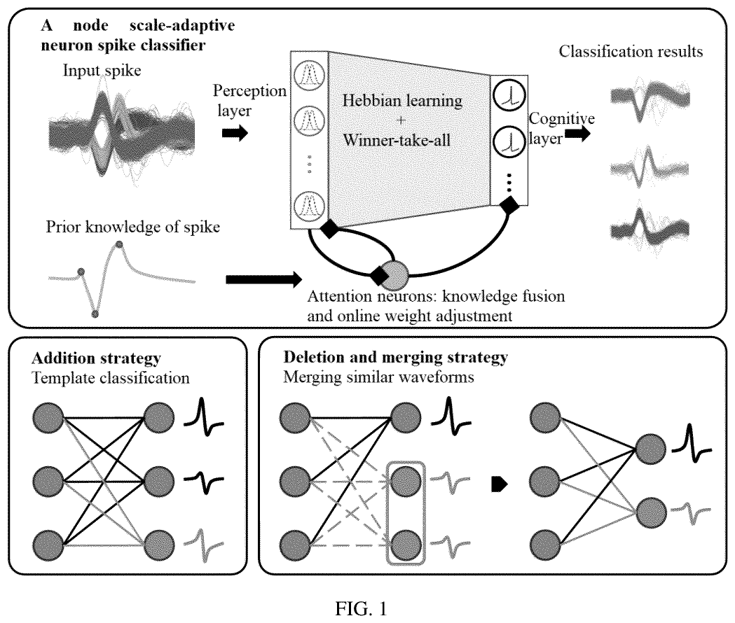

As shown in the FIG. 1, a node scale-adaptive neuron spike sorting method based on neuromorphic computing, comprising the following steps:
1. preprocessing electroencephalogram: obtaining original electroencephalogram, removing low-frequency local field potentials through band-pass filter, the 3rd order Butterworth filter with a band-pass frequency of [300 Hz, 3000 Hz] was used. The signal is finally standardized to the range [−1,1].
2. detecting the candidate spike: performing the detection of the candidate spike on each signal channel, the present invention used the nonlinear energy operator (NEO) ψ to calculate the energy intensity of each position in the discrete signal, the formula is:

$$ψ [ x ( n ) ] = {x}^{2} ( n ) - x ( n + 1 ) \cdot x ( n - 1 )$$

wherein, x(n) is the sampling point of the n time waveform. Here, the threshold is set to 0.05, and the first 25 and the last 38 energy operators that exceed the threshold are considered as candidate spike at a total of 64 time points when the window is cut off.
3. aligning spike: spike alignment refers to aligning each peak with its maximum amplitude point (in some cases, the maximum value of the peak may be the minimum value of the waveform). Due to the fact that the peak of the waveform only reaches a very short time and is usually located between the time points of signal sampling, the peak of the waveform cannot be accurately measured. To avoid peak misalignment caused by low sampling, the spike maximum peak position is interpolated using a cubic spline waveform. After realigning, for the convenience of subsequent calculations, a 256 length signal is intercepted with a peak position of 100, and all points are scaled 100 times as network inputs.
4. prior knowledge modifying: spike waveforms at different time points carry distinct physiological significance, and certain characteristic points within the waveforms often exhibit statistical regularities. By introducing the pre-hyperpolarization peak and post-hyperpolarization peak times and action potential width as key features for evaluating neural activity, the input spike waveforms are modified, the waveform modification masking formula Gₜ is defined as:

$${G}_{t} = \{ \begin{matrix}\frac{{l}_{AP} - {t}_{A 1} + t}{{l}_{AP}} , & {t}_{A 1} - {l}_{AP} \leq t < {t}_{A 1} , \\ 1 , & {t}_{A 1} \leq t \leq {t}_{A 2} , \\ 1 - \frac{t - {l}_{A 2}}{{l}_{TP}} , & {t}_{A 2} < t \leq {t}_{A 2} + {l}_{TP} , \\ 0 , & other locations .\end{matrix}$$

among them, l_{AP} represents the action potential width, t_{A1} and t_{A2} are the time points of the pre-hyperpolarization peak and post-hyperpolarization peak, respectively, and l_{TP} denotes the trough-to-peak duration of the spike.
5. inputting signal field encoding: the neural signal transmitted within the sliding time window before the current time point is encoded into a pulse signal and transmitted to the subsequent spiking or pulse neural network. The network layer of this layer is called the “perception layer”, which maps continuous input signals to a group of pulse sequences in the form of Gaussian Receptive field coding. This specific neural coding technology is an extension of the sorting encoder, which allows the vector of real value elements to be mapped to a series of pulse sequences. The Receptive field allows encoding continuous values by using a collection of neurons with overlapping sensitivity profiles.

The form of Gaussian Receptive field coding is as follows:

$${J}_{( t , m , n )} = {𝕡}_{μ , θ} ( {S}_{t} )$$

wherein, μ is the central position of neurons in the Receptive field, after normalizing the input, the bounds of the Receptive field is set to [−200,200], thus the range of m is [1,22]; θ is the width of neurons in the Receptive field, θ is set to 10; Sₜ is the signal sequence at time t, with a length of 64; J_{(t,m,n)} is the pulse firing of the neurons (m, n) in the perception layer at time t, and [symbol] is the Poisson process of the Gaussian Receptive field.
6. spiking or pulse neural network classification: comprising two layers, the first layer is neurons in the perception layer, the size of the perception layer is 22×256. The second layer is the cognitive layer, which connects the first layer neurons in a fully connected manner and dynamically updates them, here, the size of the cognitive layer is initialized to 0. The cognitive layer follows the winner-take-all (WTA) mechanism, when a neuron is activated, other neurons are suppressed and not updated.

Neuron ϵ is an integral firing (IF) neuron, the selection of neurons in the cognitive layer based on the winner-take-all mechanism is:

$$\dot{ϵ} = {max}_{ϵ} z ( ϵ , t )$$

\dot{ϵ} is the neurons in the cognitive layer for selected execution updates.

In the initial state, all weight values are initialized. Each neuron in the cognitive layer is fully connected to the perception layer. Every time the output neuron triggers a threshold, the Hebb learning rules are applied to the input synapses. Using this rule, the connection synaptic weigh \hat{ω} between two layers is updated through constant values τ₊ or τ₋:

$${\hat{ω}}_{t + 1} = STDP ( {ω}_{t} , {τ}_{+} , {τ}_{-} )$$

wherein, τ₊=0.2, τ₋=0.1.
7. nodes addition and deletion strategy updating: the addition strategy and deletion-merging strategy as shown in FIG. 1, when the perception layer node addition strategy is implemented, if the difference between the masked waveform of the input spike and the stored waveform in the network is smaller than the similarity threshold Thsim, a new node is added to the perception layer; when the perception layer node deletion and merging strategy is implemented, if the difference between stored waveforms in the network is smaller than the similarity threshold Thsim, the two nodes are merged.

When updating thresholds during perception layer node strategy adjustments, the similarity threshold Thₛᵢₘ is updated as:

$${Th}_{sim} = α \cdot ( 1 + floor ( \frac{K}{β} ) )$$

among them, α is the scaling control coefficient, β is the waveform count control coefficient, and K is the input spike waveform iteration count;
an output threshold [symbol] ₒᵤₜₚᵤₜ is updated as:

$${output}_{} = {Th}_{output} + sign ( {z}_{( t , ζ )} - {Th}_{output} ) \cdot ( 1 + floor ( \frac{K}{β} ) )$$

where [symbol] ₒᵤₜₚᵤₜ is the updated threshold for the next deletion strategy iteration, z_{(t,ψ)} is the voltage value of node ω in the perception layer at time t, β is the waveform count control coefficient 10, and K is the input spike waveform iteration count.
8. comparison results: comparing the obtained time stamp sequence with real labels to verify the performance of the method.

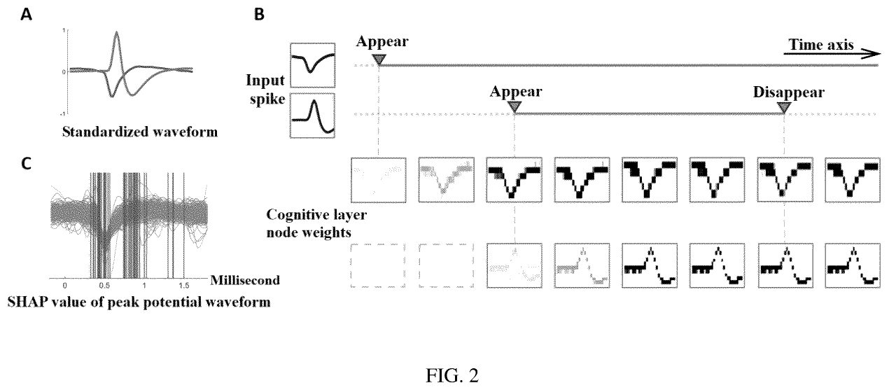

To demonstrate that our method achieves minimal computational hardware scale while fulfilling computational tasks, we examined the variations in network weights and nodes. As shown in FIG. 2, in this simulated dataset, the blue spike trains persist throughout the entire recording period, while the orange spike trains fire only for a limited duration during data acquisition. As shown by the changes in connection weights corresponding to nodes, the method automatically adds nodes in response to the emergence of spikes. Furthermore, the SHAP (SHapley Additive explanations) values of spike waveforms align with neuroscience prior knowledge, demonstrating that the method can dynamically adjust the network scale by tracking neuronal changes.

The above embodiments provide a detailed explanation of the technical solution and beneficial effects of the present invention. It should be understood that the above are only specific embodiments of the present invention and are not intended to limit the present invention. Any modifications, supplements, and equivalent replacements made within the scope of the principles of the present invention should be included in the scope of protection of the present invention.

1. A node scale-adaptive neuron spike sorting method based on neuromorphic computing, comprising the following steps:
(1) obtaining original multi-channel neural signals, removing low-frequency local field potentials through a band-pass filter, and performing whitening preprocessing and artifact removal on each channel's neural signals;
(2) detecting and aligning candidate spike on each signal channel, specifically by using a nonlinear energy operator to calculate the energy intensity of each position in the discrete signal, a time window exceeding the threshold is determined as a candidate spike, and then aligning the candidate spike based on the spike position;
(3) constructing a spiking neural network framework, the framework comprises a two-layer spiking neural network and an attention neuron node; wherein, the first layer of the spiking neural network is a perception layer and the second layer of the spiking neural network is a cognitive layer, each neuron on the two-layer connects in a fully connected manner and dynamically updates the connecting synapses; the attention neuron is unidirectionally connected to control all neurons in the network;
(4) inputting the aligning candidate spike potentials into the spiking neural network framework, wherein the perception layer of the spiking neural network is used to spike code the candidate spike, and the discrete signal of each time point of the candidate spike is mapped to a group of spike sequences in the form of Gaussian Receptive field coding;
wherein the neurons on the cognitive layer respond to different pulse sequence inputs, and update the connecting synapses between the activated neurons with the corresponding neurons in the perception layer based on the winner-take-all mechanism; when the cumulative voltage of neurons in the cognitive layer exceeds the voltage threshold, the pulse sequences are output as a time stamp sequence in response to the action potential of different cells;
wherein the attention neuron node responds to the waveform prior knowledge of input candidate spike, modifies the waveform of input candidate spike, and controls the threshold changes of the addition strategy, deletion and merging strategy of perception layer nodes;
(5) for the original neural signals corresponding to the time stamp sequence, dividing the spike and noise according to a threshold, and cognitive layer nodes dynamically updating the threshold by spiking waveform prior knowledge, and each channel reconstructs waveforms from different cells based on the time stamp sequence output by the spiking neural network.

2. The node scale-adaptive neuron spike sorting method based on neuromorphic computing according to claim 1, wherein, in the step (1), the band-pass filter adopts a 3rd order Butterworth filter with a band-pass frequency of 300-3000 Hz.

3. The node scale-adaptive neuron spike sorting method based on neuromorphic computing according to claim 1, wherein, in the step (2), using a nonlinear energy operator to calculate the energy intensity of each position in the discrete signal, the formula is:
$$ψ [ x ( n ) ] = {x}^{2} ( n ) - x ( n + 1 ) \cdot x ( n - 1 )$$
wherein, x(n) is the sampling point of the n time waveform.

4. The node scale-adaptive neuron spike sorting method based on neuromorphic computing according to claim 1, wherein, in the step (2), when aligning the candidate spike based on the spike position, the spike maximum peak position is first interpolated through upsampling, and after realignment, the waveform is downsampled to its original length.

5. The node scale-adaptive neuron spike sorting method based on neuromorphic computing according to claim 1, wherein, in the step (4), the form of Gaussian Receptive field coding is as follows:
$${J}_{( t , m , n )} = {𝕡}_{μ , θ} ( {S}_{t} )$$
wherein, μ is the central position of neurons in the Receptive field, θ is the width of neurons in the Receptive field, Sₜ is the signal sequence at time t, J_{(t,m,n)} is the pulse firing of the neurons (m, n) in the perception layer at time t, and [symbol] is the Poisson process of the Gaussian Receptive field.

6. The node scale-adaptive neuron spike sorting method based on neuromorphic computing according to claim 1, wherein, in the step (4), the winner-take-all mechanism is: when a neuron is activated, other neurons are suppressed and not updated, only the weight of connecting synapses between the activated neuron with the neurons in the perception layer is enhanced or reduced;
updating the connecting synapses between the activated neurons with the corresponding neurons in the perception layer, the neuron selection method is as follows:
$$\dot{ϵ} = {max}_{ϵ} z ( ϵ , t )$$
wherein, \dot{ϵ} is the neurons in the cognitive layer for selected execution updates, z(ϵ, t) is the voltage value of neurons in the cognitive layer at time t;
a weight update method of the connecting synapses between the two layers is as follows:
$${\hat{ω}}_{t + 1} = STDP ( {ω}_{t} , {τ}_{+} , {τ}_{-} )$$
wherein, \hat{ω}ₜ₊₁ is the synaptic weight at t+1 time after update, ωₜ is the synaptic weight at time t before the update, τ₊ is the constant for postsynapses firing, τ₋ is the constant for presynapses firing.

7. The node scale-adaptive neuron spike sorting method based on neuromorphic computing according to claim 1, wherein, in the step (4), the attention neuron node responds to the waveform prior knowledge of input candidate spike, modifies the waveform of input candidate spike, a generating method for a waveform masking [symbol] ₜ is as follows:
$${ℳ}_{t} = {S}_{t} \cdot {G}_{t}$$
among them, Sₜ represents the signal sequence at time t, Gₜ denotes the waveform modification mask; the generation method of the waveform modification mask Gₜ is as follows:
$${G}_{t} = \{ \begin{matrix}\frac{{l}_{AP} - {t}_{A 1} + t}{{l}_{AP}} , & {t}_{A 1} - {l}_{AP} \leq t < {t}_{A 1} , \\ 1 , & {t}_{A 1} \leq t \leq {t}_{A 2} , \\ 1 - \frac{t - {l}_{A 2}}{{l}_{TP}} , & {t}_{A 2} < t \leq {t}_{A 2} + {l}_{TP} , \\ 0 , & other locations ;\end{matrix}$$
among them, l_{AP} represents the action potential width, t_{A1} and t_{A2} are the time points of the pre-hyperpolarization peak and post-hyperpolarization peak, respectively, and l_{TP} denotes the trough-to-peak duration of the spike.

8. The node scale-adaptive neuron spike sorting method based on neuromorphic computing according to claim 1, wherein, in the step (4), when implementing the addition strategy of perception layer nodes: if the difference between the masked waveform of the input spike and the stored waveform in the network is smaller than the similarity threshold Thₛᵢₘ, a new node is added to the perception layer, the perception layer node update comparison method is:
$$\frac{‖ {ℳ}_{t} - ( Φ ⋂ {ω}_{ϵ} ) ‖}{‖ Φ ⋂ {ω}_{ϵ} ‖} < {Th}_{sim}$$
among them, [symbol] ₜ is the masked waveform, ω_{ϵ} represents the connection weights between the selected node ϵ in the perception layer and the previous layer, and Φ is an all-ones matrix with the same dimensions as the weight matrix.

9. The node scale-adaptive neuron spike sorting method based on neuromorphic computing according to claim 8, wherein, in the step (4), when implementing the deletion and merging strategy of perception layer nodes: if the difference between stored waveforms in the network is smaller than the similarity threshold Thₛᵢₘ, the two nodes are merged, the perception layer node update comparison method is:
$$\frac{‖ {ω}_{i} - {ω}_{j} ‖}{‖ \sqrt{{ω}_{i}} ‖ ‖ \sqrt{{ω}_{j}} ‖} < {Th}_{sim}$$
among them, ωᵢ and ωⱼ are the connection weights corresponding to distinct nodes in the perception layer.

10. The node scale-adaptive neuron spike sorting method based on neuromorphic computing according to claim 9, wherein, in the step (4), when updating thresholds during perception layer node strategy adjustments, the similarity threshold Thₛᵢₘ is updated as:
$${Th}_{sim} = α \cdot ( 1 + floor ( \frac{K}{β} ) )$$
among them, α is the scaling control coefficient, β is the waveform count control coefficient, and K is the input spike waveform iteration count;
an output threshold [symbol] ₒᵤₜₚᵤₜ is updated as:
$${output}_{} = {Th}_{output} + sign ( {z}_{( t , ζ )} - {Th}_{output} ) \cdot ( 1 + floor ( \frac{K}{β} ) )$$
where [symbol] ₒᵤₜₚᵤₜ is the updated threshold for the next deletion strategy iteration, z_{(t,ζ)} is the voltage value of node ζ in the perception layer at time t, β is the waveform count control coefficient, and K is the input spike waveform iteration count.
